SEQUENCE LISTING (55 sequences)

SEQ ID NO: 1 - protein, 1129 aa, Artificial Sequence
  Synthetic (AacC2c1)
MAVKSIKVKLRLDDMPEIRAGLWKLHKEVNAGVRYYTEWLSLLRQENLYRRSPNGDGEQECDKTAEECKAELLERLRARQVENGHRGPAGSDDELLQLARQLYELLVPQAIGAKGDAQQIARKFLSPLADKDAVGGLGIAKAGNKPRWVRMREAGEPGWEEEKEKAETRKSADRTADVLRALADFGLKPLMRVYTDSEMSSVEWKPLRKGQAVRTWDRDMFQQAIERMMSWESWNQRVGQEYAKLVEQKNRFEQKNFVGQEHLVHLVNQLQQDMKEASPGLESKEQTAHYVTGRALRGSDKVFEKWGKLAPDAPFDLYDAEIKNVQRRNTRRFGSHDLFAKLAEPEYQALWREDASFLTRYAVYNSILRKLNHAKMFATFTLPDATAHPIWTRFDKLGGNLHQYTFLFNEFGERRHAIRFHKLLKVENGVAREVDDVTVPISMSEQLDNLLPRDPNEPIALYFRDYGAEQHFTGEFGGAKIQCRRDQLAHMHRRRGARDVYLNVSVRVQSQSEARGERRPPYAAVFRLVGDNHRAFVHFDKLSDYLAEHPDDGKLGSEGLLSGLRVMSVDLGLRTSASISVFRVARKDELKPNSKGRVPFFFPIKGNDNLVAVHERSQLLKLPGETESKDLRAIREERQRTLRQLRTQLAYLRLLVRCGSEDVGRRERSWAKLIEQPVDAANHMTPDWREAFENELQKLKSLHGICSDKEWMDAVYESVRRVWRHMGKQVRDWRKDVRSGERPKIRGYAKDVVGGNSIEQIEYLERQYKFLKSWSFFGKVSGQVIRAEKGSRFAITLREHIDHAKEDRLKKLADRIIMEALGYVYALDERGKGKWVAKYPPCQLILLEELSEYQFNNDRPPSENNQLMQWSHRGVFQELINQAQVHDLLVGTMYAAFSSRFDARTGAPGIRCRRVPARCTQEHNPEPFPWWLNKFVVEHTLDACPLRADDLIPTGEGEIFVSPFSAEEGDFHQIHADLNAAQNLQQRLWSDFDISQIRLRCDWGEVDGELVLIPRLTGKRTADSYSNKVFYTNTGVTYYERERGKKRRKVFAQEKLSEEEAELLVEADEAREKSVVLMRDPSGIINRGNWTRQKEFWSMVNQRIEGYLVKQIRSRVPLQDSACENTGDI

SEQ ID NO: 2 - RNA, 91 nt, Artificial Sequence
  Synthetic (sgRNA for AacC2c1, wherein each n is independently selected from a, u, c, and g)
gucuagaggacagaauuuuucaacgggugugccaauggccacuuuccagguggcaaagcccguugagcuucucaaaucugagaaguggnnn

SEQ ID NO: 3 - RNA, 91 nt, Artificial Sequence
  Synthetic (sgRNA for AacC2c1)
gucuagaggacagaauuuuucaacgggugugccaauggccacuuuccagguggcaaagcccguugagcuucucaaaucugagaaguggcac

SEQ ID NO: 4 - protein, 7 aa, Artificial Sequence
  Synthetic (NLS)
PKKKRKV

SEQ ID NO: 5 - DNA, 60 bp, Artificial Sequence
  Synthetic (CMV-AacC2c1-CN-1F-Hind3 primer)
gctggctagcgtttaaacttaagcttggtaccgccaccatggcagtgaaaagcatcaaag

SEQ ID NO: 6 - DNA, 39 bp, Artificial Sequence
  Synthetic (CMV-AacC2c1-CN-1R-BamH1 primer)
aacatcgtatgggtaggatcctcaggcgtagtcgggcac

SEQ ID NO: 7 - DNA, 242 bp, Artificial Sequence
  Synthetic (Aac-sgRNA cloning vector)
ggtctagaggacagaatttttcaacgggtgtgccaatggccactttccaggtggcaaagcccgttgagcttctcaaatctgagaagtggcactgagacgggatccatccgtctccttttttccagatctcctgtcttaaaaagttgcccacacggttaccggtgaaaggtccaccgtttcgggcaactcgaagagtttagactcttcaccgtgactctgccctaggtaggcagaggaaaaaa

SEQ ID NO: 8 - DNA, 20 bp, Artificial Sequence
  Synthetic (hHPRT1-E2-1F primer)
gtgctgggattacacgtgtg

SEQ ID NO: 9 - DNA, 20 bp, Artificial Sequence
  Synthetic (hHPRT1-E2-1R primer)
tgattcagccccagtccatt

SEQ ID NO: 10 - DNA, 57 bp, Artificial Sequence
  Synthetic (hHPRT1-E2-deepF primer)
acactctttccctacacgacgctcttccgatcttgtttgtatcctgtaatgctctca

SEQ ID NO: 11 - DNA, 58 bp, Artificial Sequence
  Synthetic (hHPRT1-E2-deepR primer)
gtgactggagttcagacgtgtgctcttccgatctcaaatagcaagtactcagaacagc

SEQ ID NO: 12 - DNA, 20 bp, Artificial Sequence
  Synthetic (hHPRT1-E3-1F primer)
agttcactccagcctcaaca

SEQ ID NO: 13 - DNA, 20 bp, Artificial Sequence
  Synthetic (hHPRT1-E3-1R primer)
cccaagtcccaacagcaatt

SEQ ID NO: 14 - DNA, 58 bp, Artificial Sequence
  Synthetic (hHPRT1-E3-deepF)
acactctttccctacacgacgctcttccgatctggaagtttaatgactaagaggtgtt

SEQ ID NO: 15 - DNA, 60 bp, Artificial Sequence
  Synthetic (hHPRT1-E3-deepR primer)
gtgactggagttcagacgtgtgctcttccgatctagctcttcagtctgataaaatctaca

SEQ ID NO: 16 - DNA, 20 bp, Artificial Sequence
  Synthetic (hCCR5-1-1F primer)
gtttgcattcatggagggca

SEQ ID NO: 17 - DNA, 20 bp, Artificial Sequence
  Synthetic (hCCR5-1-1R primer)
gaccagccccaagatgacta

SEQ ID NO: 18 - DNA, 56 bp, Artificial Sequence
  Synthetic (hCCR5-1-deepF primer)
acactctttccctacacgacgctcttccgatctcatcctcatcctgataaactgca

SEQ ID NO: 19 - DNA, 54 bp, Artificial Sequence
  Synthetic (hCCR5-1-deepR primer)
gtgactggagttcagacgtgtgctcttccgatctcaaacacagcatggacgaca

SEQ ID NO: 20 - DNA, 20 bp, Artificial Sequence
  Synthetic (hCCR5-2-1F primer)
ccttctgggctcactatgct

SEQ ID NO: 21 - DNA, 20 bp, Artificial Sequence
  Synthetic (hCCR5-2-1R primer)
gcgtcatcccaagagtctct

SEQ ID NO: 22 - DNA, 53 bp, Artificial Sequence
  Synthetic (hCCR5-2-deepF primer)
acactctttccctacacgacgctcttccgatcttttgcgtctctcccaggaat

SEQ ID NO: 23 - DNA, 56 bp, Artificial Sequence
  Synthetic (hCCR5-2-deepR primer)
gtgactggagttcagacgtgtgctcttccgatctgatggtgaagataagcctcaca

SEQ ID NO: 24 - DNA, 20 bp, Artificial Sequence
  Synthetic (hDNMT1-1-1F primer)
ttgggaatgtgggtactgct

SEQ ID NO: 25 - DNA, 20 bp, Artificial Sequence
  Synthetic (hDNMT1-1-1R primer)
cagaggttacagtgagccga

SEQ ID NO: 26 - DNA, 56 bp, Artificial Sequence
  Synthetic (hDNMT1-1-deepF primer)
acactctttccctacacgacgctcttccgatcttgtgtttccttgttctctgacac

SEQ ID NO: 27 - DNA, 54 bp, Artificial Sequence
  Synthetic (hDNMT1-1-deepR
gtgactggagttcagacgtgtgctcttccgatctgtttggaaaggggtttgggg

SEQ ID NO: 28 - DNA, 20 bp, Artificial Sequence
  Synthetic (hDNMT1-2-1F primer)
taggcatgtaccaccacacc

SEQ ID NO: 29 - DNA, 20 bp, Artificial Sequence
  Synthetic (hDNMT1-2-1R primer)
tcagtgcccaccgataatca

SEQ ID NO: 30 - DNA, 53 bp, Artificial Sequence
  Synthetic (hDNMT1-2-deepF primer)
acactctttccctacacgacgctcttccgatcttgtcaagtgggtgcttctct

SEQ ID NO: 31 - DNA, 55 bp, Artificial Sequence
  Synthetic (hDNMT1-2-deepR primer)
gtgactggagttcagacgtgtgctcttccgatcttgtagaacttgttgacccgga

SEQ ID NO: 32 - DNA, 24 bp, Artificial Sequence
  Synthetic (AacC2c1-HPRT1-TS1)
ttttgcatacctaatcattatgct

SEQ ID NO: 33 - DNA, 24 bp, Artificial Sequence
  Synthetic (AacC2c1-HPRT1-TS2)
attatgctgaggatttggaaaggg

SEQ ID NO: 34 - DNA, 24 bp, Artificial Sequence
  Synthetic (AacC2c1-HPRT1-TS3)
attaggtatgcaaaataaatcaag

SEQ ID NO: 35 - DNA, 24 bp, Artificial Sequence
  Synthetic (AacC2c1-HPRT1-TS4)
tttccaaatcctcagcataatgat

SEQ ID NO: 36 - DNA, 24 bp, Artificial Sequence
  Synthetic (AacC2c1-HPRT1-TS5)
attgtagccctctgtgtgctcaag

SEQ ID NO: 37 - DNA, 24 bp, Artificial Sequence
  Synthetic (AacC2c1-HPRT1-TS6)
tttgctgacctgctggattacatc

SEQ ID NO: 38 - DNA, 24 bp, Artificial Sequence
  Synthetic (AacC2c1-HPRT1-TS7)
cttgagcacacagagggctacaat

SEQ ID NO: 39 - DNA, 24 bp, Artificial Sequence
  Synthetic (AacC2c1-HPRT1-TS8)
tttgatgtaatccagcaggtcagc

SEQ ID NO: 40 - DNA, 24 bp, Artificial Sequence
  Synthetic (AacC2c1-CCR5-TS1)
tttccttcttactgtccccttctg

SEQ ID NO: 41 - DNA, 24 bp, Artificial Sequence
  Synthetic (AacC2c1-CCR5-TS2)
cttactgtccccttctgggctcac

SEQ ID NO: 42 - DNA, 24 bp, Artificial Sequence
  Synthetic (AacC2c1-CCR5-TS3)
atttccaaagtcccactgggcggc

SEQ ID NO: 43 - DNA, 24 bp, Artificial Sequence
  Synthetic (AacC2c1-CCR5-TS4)
gttgacacattgtatttccaaagt

SEQ ID NO: 44 - DNA, 24 bp, Artificial Sequence
  Synthetic (AacC2c1-CCR5-TS5)
tttccagacattaaagatagtcat

SEQ ID NO: 45 - DNA, 24 bp, Artificial Sequence
  Synthetic (AacC2c1-CCR5-TS6)
attaaagatagtcatcttggggct

SEQ ID NO: 46 - DNA, 24 bp, Artificial Sequence
  Synthetic (AacC2c1-CCR5-TS7)
cttgtcatggtcatctgctactcg

SEQ ID NO: 47 - DNA, 24 bp, Artificial Sequence
  Synthetic (AacC2c1-CCR5-TS8)
gtttttaggattcccgagtagcag

SEQ ID NO: 48 - DNA, 24 bp, Artificial Sequence
  Synthetic (AacC2c1-DNMT1-TS1)
tttgtccttggagaacggtgctca

SEQ ID NO: 49 - DNA, 24 bp, Artificial Sequence
  Synthetic (AacC2c1-DNMT1-TS2)
gttctccaaggacaaatctttatt

SEQ ID NO: 50 - DNA, 24 bp, Artificial Sequence
  Synthetic (AacC2c1-DNMT1-TS3)
cttacaaccgggaagtgaatggac

SEQ ID NO: 51 - DNA, 24 bp, Artificial Sequence
  Synthetic (AacC2c1-DNMT1-TS4)
attcacttcccggttgtaagcatg

SEQ ID NO: 52 - DNA, 24 bp, Artificial Sequence
  Synthetic (AacC2c1-DNMT1-TS5)
tttccggtagtgctctgggtacag

SEQ ID NO: 53 - DNA, 24 bp, Artificial Sequence
  Synthetic (AacC2c1-DNMT1-TS6)
gttgctgcctttgatgtagtcgga

SEQ ID NO: 54 - DNA, 24 bp, Artificial Sequence
  Synthetic (AacC2c1-DNMT1-TS7)
attggccggatcaaagagatcttc

SEQ ID NO: 55 - DNA, 24 bp, Artificial Sequence
  Synthetic (AacC2c1-DNMT1-TS8)
cttctgtcccaagaagagcaacgg

(19) United States
(12) Patent Application Publication
(10) Pub. No.: US 2020/0131487 A1
(43) Pub. Date: Apr. 30, 2020

(54) COMPOSITION CONTAINING C2CL ENDONUCLEASE FOR DIELECTRIC CALIBRATION AND METHOD FOR DIELECTRIC CALIBRATION USING SAME

(71) Applicant: INSTITUTE FOR BASIC SCIENCE, Daejeon (KR)

(72) Inventors: Jin-Soo KIM, Seoul (KR); Jung Eun KIM, Seoul (KR)

(21) Appl. No.: 16/491,208

(22) PCT Filed: Mar. 6, 2018
(86) PCT No.: PCT/KR2018/002654

§ 371 Date: Dec. 5, 2019

(30) Foreign Application Priority Data
Mar. 6, 2017 (KR) 10-2017-00258567

(51) Int. Cl. C12N 9/22; C12N 15/11; C12N 15/90; A01K 67/027

(52) U.S. Cl. CPC C12N 9/22; C12N 15/11; C12N 15/907; A01K 2217/07; C12N 2800/80; C12N 2310/20; A01K 67/0275

(57) ABSTRACT

Provided is a dielectric calibration technique using C2c1 endonuclease. The dielectric calibration technique is characterized by being especially applicable to eukaryotic cells, for example, to mammalian cells.

### TECHNICAL FIELD

The disclosure pertains to a genome-editing technique using C2c1 endonuclease and, specifically, to a genome-editing composition comprising C2c1 endonuclease and guide RNA and a genome-editing method using the same.

### BACKGROUND ART

The CRISPR-Cas system is a protecting mechanism, found in bacteria and archaea, which confers resistance to genetic elements. The CRISPR-Cas system is characterized by recognizing and cleaving specific target sequences of DNA or RNA by using a guide RNA capable of complementarily binding to foreign DNA or RNA and a Cas effector protein serving as a nuclease. Thanks to the characteristics, the system has recently been used as a gene editing technique capable of editing genes of higher organisms and thus has a wide variety of applications in basic and applied scientific fields.

The most widely used CRIPR gene-editing technique is based on the type 2 CRISPR-Cas9 system found in Streptococcus pyogenes. The CRISPR-Cas9 system derived from S. pyogenes is generally effective in the sense of the ability to induce gene editing at desired target sites at high efficiency, but within the limitation that the presence of 5′-NGG PAM sequence is required for the target sequence thereof. As one of the strategies for overcoming the limitation, extensive studies have been conducted into the plausibility of utilizing novel CRISPR-Cas systems or other types of gene editing systems from different species.

### DETAILED DESCRIPTION OF THE INVENTION

### Technical Problem

An aspect provides a genome-editing composition comprising a C2c1-CRISPR system, a gene encoding the same (DNA (cDNA, rDNA, etc.) or RNA (mRNA, etc.)), or an expression vector carrying the gene.

Another aspect provides a genome-editing method comprising a step of introducing the genome-editing composition into a cell or an organism.

Another aspect provides a genetically modified cell obtained using the genome-editing composition.

Another aspect provides a genetically modified organism obtained from the genetically modified cell.

Another aspect provides a method for production of a transgenic animal, the method comprising a step of transplanting into an oviduct of a mammalian foster mother a mammalian embryo having the genome-editing composition introduced thereinto.

### Technical Solution

The C2c1-CRISPR system is a type VB CRISPR-Cas system found recently. As opposed to the SpCas9 system most utilized conventionally, the C2c1-CRISPR system is characterized by containing C2c1 protein, instead of Cas9, as an effector protein serving as a nuclease, recognizing 5′-TTN PAM sequence instead of than 5′-TTN PAM sequence, and forming a sticky end rather than a blunt end upon DNA cleavage.

Proposed in this description is a technique employing a C2c1 endonuclease for gene editing in eukaryotic cells such as human cells.

An aspect provides a genome-editing composition comprising a C2c1-CRISPR system, a gene encoding the same (DNA (cDNA, rDNA, etc.) or RNA (mRNA, etc.)), or an expression vector carrying the gene.

The C2c1-CRISPR system may contain a C2c1 protein (e.g., AacC2c1 protein) and a guide RNA (e.g., single-guide RNA (sgRNA)) capable of hybridizing with (having a complementary nucleotide sequence to) a target site of a target gene.

Accordingly, the genome-editing composition may comprise (1) C2c1 endonuclease, a gene coding therefor, or an expression vector carrying the gene, and (2) a guide RNA, a DNA coding therefor, or an expression vector carrying the same.

Another aspect provides a genome-editing method comprising a step of introducing (administering or injecting) the genome-editing composition into a cell or an organism. The cell may be a cell (e.g., eukaryotic cell) isolated from a living organism and the organism may be an eukaryotic organism (e.g., mammal) exclusive or inclusive of a human being.

The cell may be an eukaryotic cell such as an eukaryotic animal, an eukaryotic plant cell, or the like, and the organism may be an eukaryotic organism such as an eukaryotic animal or an eukaryotic plant.

Another aspect provides a genetically modified cell obtained using the genome-editing composition. The cell may be an eukaryotic cell. The cell may be a cell from an eukaryotic animal. In an embodiment, the cell may be a cell from a mammal inclusive or exclusive of a human being.

Another aspect provides a method for production of a transgenic animal, the method comprising a step of transplanting into an oviduct of a mammalian foster mother a mammalian embryo having the genome-editing composition introduced thereinto. The mammalian animal (foster mother) having the embryo transplanted into the oviduct thereof may be the same species as in the mammalian animal from which the embryo is derived.

Another aspect provides a genetically modified organism obtained from the genetically modified cell. The genetically modified organism may be generated by a method for generating a transgenic animal. The animal may be an eukaryotic animal, such as a mammal inclusive or exclusive of a human being.

The C2c1-CRISPR endonuclease is found in various bacterial species. A representative example is Alicyclobacillus acidoterrestris-derived C2c1-CRISPR endonuclease (hereinafter referred to as “AacC2c1”). Wild-type (WT) AacC2c1 is composed of 1129 amino acids (see SEQ ID NO: 1) and may be a protein encoded by the nucleotide sequence provided in the website https://www.addgene.org/browse/sequence/125305/ (a region from position 1771 to position 5160).

As used herein, the term “guide RNA” refers to an RNA that includes a targeting sequence hybridizable with a specific base sequence (target sequence) of a target site in a target gene and functions to associate with a nuclease, such as Cad 1 protein, etc., and guide the nuclease to a target gene (or target site) in vitro or in vivo (or cells).

The guide RNA may be suitably selected depending on kinds of the nuclease to be complexed therewith and/or origin microorganisms thereof.

For example, the guide RNA may be at least one selected from the group consisting of:

CRISPR RNA (crRNA) including a region (targeting sequence) hybridizable with a target sequence;

trans-activating crRNA (tracrRNA) including a region interacting with a nuclease such as C2c1 protein, etc.; and

single guide RNA (sgRNA) in which main regions of crRNA and tracrRNA (e.g., a crRNA region including a targeting sequence or a tracrRNA region interacting with nuclease) are fused to each other.

In detail, the guide RNA may be a dual RNA including CRISPR RNA (crRNA) and trans-activating crRNA (tracrRNA) or a single guide RNA (sgRNA) including main regions of crRNA and tracrRNA.

The sgRNA may include a region (named “spacer region”, “target DNA recognition sequence”, “base pairing region”, etc.) having a complementary sequence (targeting sequence) to a target sequence in a target gene (target site), and a hairpin structure for binding to a C2c1 protein. So long as it includes main regions of crRNA and tracrRNA and a complementary sequence to a target DNA, any guide RNA can be used in the present disclosure.

For editing a target gene, for example, the C2c1 protein requires two guide RNAs, that is, a CRISPR RNA (crRNA) having a nucleotide sequence hybridizable with a target site in the target gene and a trans-activating crRNA (tracrRNA) interacting with the C2c1 protein. In this context, the crRNA and the tracrRNA may be coupled to each other to form a crRNA:tracrRNA duplex or connected to each other via a linker so that the RNAs can be used in the form of a single guide RNA (sgRNA) in which a partial double strand is formed to make a folding structure suitable for binding the C2c1 protein. In one embodiment, when AacC2c1 is used, the sgRNA may form a structure in which the entirety or a part of the crRNA having a hybridizable nucleotide sequence is connected to the entirety or a part of the tracrRNA including an interacting region with the C2c1 protein.

The guide RNA, specially, crRNA or sgRNA, includes a targeting sequence complementary to a target sequence in a target gene and may contain one or more, for example, 1-10, 1-5, or 1-3 additional nucleotides at an upstream region of crRNA or sgRNA, particularly at the 5′ end of sgRNA or the 5′ end of crRNA in dual RNA. The additional nucleotide(s) may be guanine(s) (G), but are not limited thereto.

In one embodiment, the sgRNA for AacC2c1 may be represented by the following General Sequence Formula 1:

5′-

GUCUAGAGGACAGAAUUUUUCAACGGGUGUGCCAAUGGCCACUUUCCA
GGUGGCAAAGCCCGUUGAGCUUCUCAAAUCUGAGAAGUGGX₁X₂X₃[(N)n]-3′
(5′-[SEQ ID NO: 2]-[(N)n]-3′; General Sequence Formula 1)

wherein,

the region in bold is derived from tracrRNA,

the region in italics is derived from crRNA,

X₁, X₂, and X₃ are each a nucleotide independently selected from A, U, G, and C,

(N)n is a targeting sequence hybridizable with (having a complementary sequence to) a target sequence and consisting of n nucleotides that may be the same or different and are each independently selected from A, U, G, and C, wherein n is an integer of 17-23, 18-22, or 19-21 (e.g., 20).

In an embodiment, the guide RNA is a mutant resulting from the deletion of 1-15, 5-15, 10-15, or 12-15 nucleotides from the sequence of General Sequence Formula 1. In this context, the deleted nucleotide(s) may be 1-15, 5-15, 10-15, or 12-15 (e.g., 12 or 15) nucleotide(s) selected from the nucleotides of the underlined sequence (AGCUUCUCAAA) in General Sequence Formula 1. In an embodiment, the sigRNA for AacC2c1 may be represented by General

Sequence Formula 2, below, but is not limited thereto:

5′-GUCUAGAGGACAGAAUUUUUCAACGGGUGUGCCAAUGGCCACUUUCCAGG
UGGCAAAGCCCGUUGAGCUUCUCAAAUCUGAGAAGUGGCAC[(N)n]-3′
(5′-[SEQ ID NO: 3]-[(N)n]-3′; General Sequence Formula 2).

In General Sequence Formula 2, n may be 20.

The crRNA or sgRNA may further contain 1 to 3 guanines (G) at the 5′ end thereof (that is, the 5′ end of the targeting sequence of crRNA).

The target sequence for the guide RNA may be about 17 to about 23, about 18 to about 22, or about 19 to about 21, for example, 20 consecutive nucleotides adjacent to the 5′ end of PAM (Protospacer Adjacent Motif (5′-TTN-3′ (N is A, T, G, or C)) on a target DNA.

As used herein, the term “the targeting sequence” of guide RNA, which is hybridizable with the target sequence for the guide RNA, refers to a nucleotide sequence having a sequence complementarity of 50% or higher, 60% or higher, 70% or higher, 80% or higher, 90% or higher, 95% or higher, 99% or higher, or 100% to a nucleotide sequence of a complementary strand to a DNA strand on which the target sequence exists (i.e., a PAM sequence (5′-TTN-3′ (N is A, T, G, or C))). In an embodiment, the targeting sequence of guide RNA may be a sequence complementary to and hybridizable with a nucleotide sequence on the complementary strand to the target strand on which the PAM sequence exists.

In the description, a nucleic acid sequence (target sequence) at a target site is represented by that of the strand on which a PAM sequence exists between two DNA strands in a region of a target gene. In this regard, the DNA strand to which the guide RNA couples is complementary to a strand on which a PAM sequence exists. Hence, the targeting sequence included in the guide RNA has the same nucleic acid sequence as a sequence at an on-target site, with the exception that U is employed instead of T due to the RNA property. In other words, a targeting sequence of the guide RNA and a target sequence are represented by the same nucleic acid sequence with the exception that T and U are interchanged, in the description.

The C2c1 endonuclease and the guide RNA may be in the form of:

a ribonucleic acid protein in which the C2c1 endonuclease protein and the guide RNA are complexed with each other in vitro (assembled in advance in vitro) (in this regard, injected in the form of a complex into a body or cells and transported across cell membranes and/or nuclear membranes);

a mixture of the C2c1 endonuclease or an mRNA coding therefor, and the guide RNA; or

a plasmid carrying together DNAs coding respectively for the C2c1 endonuclease protein and the guide RNA or separate plasmids respectively carrying the DNAs.

The C2c1 protein may be linked to a cell penetrating peptide and/or a protein transduction domain. The protein transduction domain may be poly-arginine or an HIV-derived TAT protein, but is not limited thereto. Because there are various kinds of the cell penetrating peptide or the protein transduction domain in addition to the stated examples, a person skilled in the art may make application of various kinds without limitations to the examples.

In addition, the C2c1 protein or a gene coding therefor may further comprise a nuclear localization signal (NLS) sequence or a nucleic acid sequence coding therefor. Therefore, an expression cassette including a C2c1 protein-encoding gene may further comprise a regulatory sequence such as a promoter sequence for expressing the C2c1 protein and/or nuclease and optionally an NLS sequence. The NLS sequence is well known in the art and may have the amino acid sequence of PKKKRKV, but is not limited thereto. Particularly when the C2c1 protein or a ribonucleic acid protein containing the same is applied to eukaryotic cells and/or eukaryotic organisma, the NLS sequence may be needed.

The C2c1 protein or the nucleic acid coding therefor may be linked to a tag for isolation and/or purification or a nucleic acid coding for the tag. For example, the tag may be selected from the group consisting of small peptide tags, such as His tag, Flag tag, S tag, etc., GST (Glutathione S-transferase) tag, and MBP (Maltose binding protein) tag, but is not limited thereto.

As used herein, the term “genome editing” means deletion and/or insertion of at least one nucleotide and/or substitution of a different nucleotide for an original nucleotide in a target gene in a genome, and/or the inactivation (dysfunction or functional loss) of the target gene.

The C2c1 protein breaks a DNA double strand at a vicinity of the PAM sequence, forming a sticky end, which results in different lengths for the two strands at the ends, because of different cutting positions on the two strands. AcsC2c1 may break a DNA double strand at a position 5-25 nt (nucleotides), 5-20 nt, 10-25 nt, 10-20 nt, or 15-20 nt distant from the PAM sequence in the 3′ direction on the PAM sequence-bearing strand (5′ or 3′ end of the nucleotide at the position; e.g., between nucleotides at positions 17 and 18 from the PAM in the 3′ direction) and at a position 15-30 nt (nucleotides), 15-25 nt, 20-30 nt, or 20-25 nt distant from a sequence complementary to the PAM sequence in the 5′ direction on the other stand (complementary to the PAM sequence-bearing strand), forming a sticky end. The break positions may vary depending on the positions of target sequences.

The cells may be prokaryotic or eukaryotic (eukaryotic animal cells or eukaryotic plant cells) and particularly may be cells from eukaryotic animals such as mammals inclusive of humans. The cells may exist in a living body or may be cells isolated from a living body.

The introduction of genes into cells may be conducted by introducing an expression vector carrying the C2c1-encoding gene and the guide RNA-encoding gene together or expression vectors respectively carrying the genes into cells in a conventional process (electroporation, lipofection, etc.) and expressing the genes therein, or by directly introducing a C2c1-guide RNA complex assembled in vitro in advance or a mixture of a C2c1 protein or mRNA coding therefor and a guide RNA into cells through electroporation, lipofection, microinjection, etc. without use of an expression vector. The introduction may be conducted in vitro for cells isolated from a living body.

As used herein,

“target gene” means a gene as an object to which base editing (or base mutation) is applied, and

“target site” or “target region” means a site or region at which a target-specific nuclease performs base editing in a target gene. In one embodiment, when the target-specific nuclease includes an RNA-guided nuclease (RNA-guided engineered nuclease; RGEN), the target site or target region is intended to be a gene site (a double strand, or any one single strand in a double strand) which is located adjacent to the 5′- and/or 3′-end of the RNA-guided nuclease-recognized sequence (PAM sequence) in a target gene and has a maximum length of about 50 bp or about 40 bp.

In one embodiment, when the target-specific nuclease includes an RNA-guided nuclease, a guide RNA containing a targeting sequence may be included together with the RNA-guided nuclease. The “targeting sequence” may be a guide RNA site including a base sequence complementary to (hybridizable with) a consecutive base sequence of about 15 to about 30 nucleotides (nt), about 15 to about 35 nt, about 17 to about 23 nt, or about 18 to about 22 nt, e.g., about 20 nt on a target site. The base sequence on a target site, complementary to the targeting sequence, is called a “target sequence”. The “target sequence” may mean a consecutive base sequence of about 15 nt to about 30 nt, about 15 nt to about 25 nt, about 17 nt to about 23 nt, or about 18 nt to about 22 nt, for example, about 20 nt located adjacent to the 5′- and/or 3′-end of a PAM sequence recognized by an RNA-guided nuclease.

### Advantageous Effects

In the description, it is first suggested that the type VB CRISPR-C2c1 system can be utilized in human genome editing. The C2c1-CRISPR derived from Alicyclobacillus acidoterrestris was representatively employed. Even though the bacterium is a thermophile optimized at high temperatures, the C2c1-CRISPR derived therefrom was found to operate at high efficiency for specific target sequences of human HPRT1 gene even in a 37° C. condition. Given, a C2c1-CRISPR system derived from mesophilic bacteria that live in a temperature condition suitable for human genome editing other than from thermophiles is expected to guarantee a novel gene editing tool capable of genome editing at higher efficiency, making contribution to expanding a genome editing platform using CRISPR systems.

### BRIEF DESCRIPTION OF THE DRAWINGS

FIGS. 1a to 1c show configurations of AacC2c1-CRISPR system expression vectors and results of the protein expression using the same, presenting schematic diagrams of 1a an AacC2c1 protein expression vector and an Aac-sgRNA cloning vector (1a) and AacC2c1 protein expression results (1b) (left panel: 1^{st} antibody, HA tag; and right panel: 1^{st} antibody, GAPDH).

FIGS. 2a to 2d show human HPRT1 gene editing results obtained by using the AacC2c1 and sgRNA expression vectors, presenting graphs of human HPRT1 target gene editing results (Indel (%)) obtained using AacC2c1 for Hela cells (2a) and HEK293T cells (2b) (Biological replicate (N=3)), 1^{st} NGS analysis results for HPRT1-TS2 and HPRT1-TS5 (2c) (PAM sequence in blue; AacC2c1 target sequences in upper cases; sequences around the AacC2c1 target in lower cases; and WT, wild type), and photographs of colony formation assay results obtained through crystal violet staining (2d) (after transfection of AacC2c1 and sgRNA expression vectors thereinto, Hela cells were seeded and subjected to 6-thioguanine (6-TG) selection. Two weeks later, colonies of the cells in which a HPRT1 gene was knocked out by mutation were identified by crystal violet staining)

FIGS. 3a to 3e show gene editing results obtained by applying AacC2c1 and sgRNA expression vectors to human CCR5 and DNMT1 genes, presenting graphs of human CCR5 target gene editing results (Indel (%)) obtained using AacC2c1 for Hela cells (3a) and HEK293T cells (3b) (Biological replicate (N=1)), graphs of human DNMT1 target gene editing results (Indel (%)) obtained using AacC2c1 or Hela cells (3c) and HEK293T cells (3d) (Biological replicate (N=1)), and 1^{st} NGS analysis results for CCR5-TS3 and DNMT1-TS4 (3e) (PAM sequence in blue; AacC2c1 target sequences in upper cases; sequences around the AacC2c1 target in lower cases; and WT, wild type).

### MODE FOR CARRYING OUT THE INVENTION

Hereinafter, the present invention will be described in detail with reference to examples. These examples are only for illustrating the present invention more specifically, and it will be apparent to those skilled in the art that the scope of the present invention is not limited by these examples.

### Example 1: Vector Construction

With pRSFDuet1-His6-Sumo-AacC2C1-CN plasmid (pRSF-Duet-1-His6-SUMO-AacC2c1 plasmid, granted by Pf. Dinshaw Patel group, was added with an NLS(PKKKRKV)-HA tag sequence at the C-terminal of AacC2c1) serving as a template, the coding sequence of AacC2c1 was amplified by PCR (CMV-AacC2c1-CN-1F-Hind3: GCTGGCTAGCGTTTAAACTTAAGCTTGGTACCGCCACCATGGCAGTGAAAAG CATCAAAG (SEQ ID NO: 5), CMV-AacC2c1-CN-1R-BamH1: AACATCGTATGGGTAGGATCCTCAGGCGTAGTCGGGCAC (SEQ ID NO: 6), phusion DNA polymerase was used), and the PCR product was inserted into pcDNA3.1 backbone vector (Invitrogen, V790-20) by Gibson cloning.

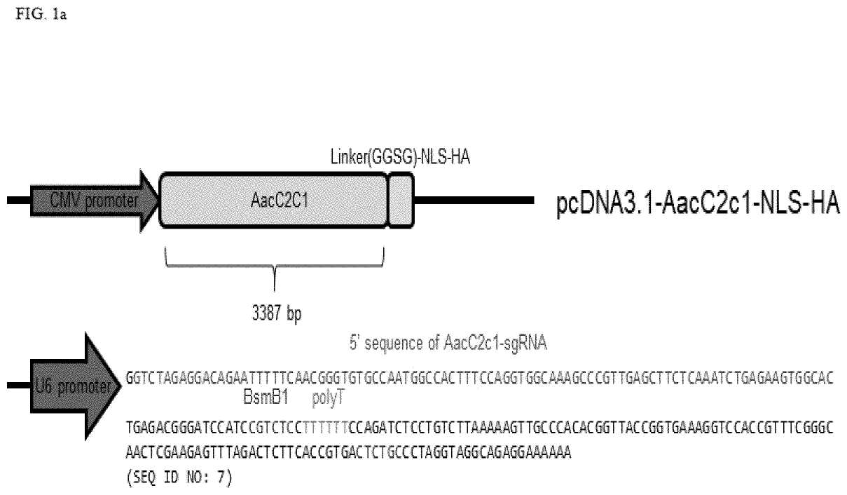

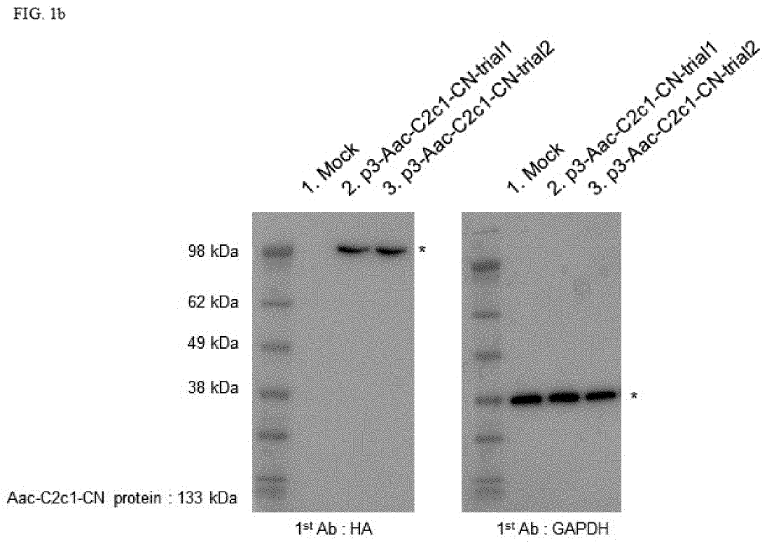

In order to be optimized for human cell expression and genome editing, the above-prepared AacC2c1 coding sequence was used, together with the CMV promoter and the nuclear localization signal (PKKKRKV), to construct an expression cassette. An AacC2c1 expression vector was constructed under the design that the AacC2c1 gene is transcribed under the CMV promoter suitable for human cell expression and the SV40 nucleus localization signal (PKKKRKV) and the HA tag are positioned at the C terminal of the AacC2c1 coding sequence (pcDNA3.1-AacC2c1-NLS-HA; FIG. 1a).

An Aac-sgRNA cloning vector was constructed in which the sgRNA binding complementarily to a target sequence was transcribed under the human U6 promoter. For Aac-sgRNA, the sequence in the 5′-terminal sequence was used in common while 20 consecutive nucleotides at the 3′-terminal site of sgRNA were substituted in accordance with target sequences (see Table 2) to construct sgRNA that acted at specific target positions (see General Sequence Formula 2). The Aac-sgRNA cloning vector was constructed on the basis of a U6-Sp-sgRNA cloning vector (see FIG. 1a).

### Example 2: Cell Test

Hela cells (ATCC) and HEK293T cells (ATCC) were both tested in 24-well-plate scales. On the day before transfection, cells were seeded. The next day, transfection was performed at 70-80% confluency. Using lipofectamine2000 (Invitrogen), 500 ng of each of the AacC2c1 and Aac-sgRNA vectors prepared in Example 1 was delivered into the cells. 72 hours after transfection, genomic DNA samples were extracted using DNeasy Blood & Tissue kit (Qiagen) and subjected to the following additional analyses.

### Example 3: Targeted Deep Sequencing

A Deep sequencing library was prepared by PCR. TruSeq HT Dual Index primers were used to assign respective I.D.s to samples, followed by analysis with the aid of MiniSeq 300 cycles kit (Illumina). Primer sequences used in the deep sequencing are summarized in Table 1, below:

TABLE 1

| | | SEQ | | |
|---|---|---|---|---|
| | Primer | ID | | |
| Locus | name | NO | Primer sequence | Target site |
| HPRT1 | hHPRT1- | 8 | gtgctgggattacacgtgtg | HPRT1-E2-TS1, |
| | E2-1F | | | HPRT1-E2-TS2, |
| | hHPRT1- | 9 | tgattcagccccagtccatt | HPRT1-E2-TS3, |
| | E2-1R | | | HPRT1-E2-TS4 |
| | hHPRT1- | 10 | ACACTCTTTCCCTACACGACGCTCTT | |
| | E2- | | uATCTtgtttgtatcctgtaatgctctca | |
| | deepF | | | |
| | hHPRT1- | 11 | GTGACTGGAGTTCAGACGTGTGCTC | |
| | E2- | | TTCCGATCTcaaatagcaagtactcagaacagc | |
| | deepR | | | |
| | hHPRT1- | 12 | agttcactccagcctcaaca | HPRT1-E2-TS5, |
| | E3-1F | | | HPRT1-E2-TS6, |
| | hHPRT1- | 13 | cccaagtcccaacagcaatt | HPRT1-E2-TS7, |
| | E3-1R | | | HPRT1-E2-TS8 |
| | hHPRT1- | 14 | ACACTCTTTCCCTACACGACGCTCTT | |
| | E3- | | CCGATCTggaagtttaatgactaagaggtgtt | |
| | deepF | | | |
| | hHPRT1- | 15 | GTGACTGGAGTTCAGACGTGTGCTC | |
| | E3- | | TTCCGATCTagctcncagtctgataaaatcta | |
| | deepR | | ca | |
| CCR5 | hCCR5- | 16 | gtttgcattcatggagggca | CCR5-TS1, |
| | 1-1F | | | CCR5-TS2, |
| | hCCR5- | 17 | gaccagccccaagatgacta | CCR5-TS3, |
| | 1-1R | | | CCR5-TS4 |
| | hCCR5- | 18 | ACACTCTTTCCCTACACGACGCTCTT | |
| | 1-deepF | | CCGATCTcatcctcatcctgataaactgca | |
| | hCCR5- | 19 | GTGACTGGAGTTCAGACGTGTGCTC | |
| | 1-deepR | | TTCCGATCTcaaacacagcatggacgaca | |
| | hCCR5- | 20 | ccttctgggctcactatgct | CCR5-TS5, |
| | 2-1F | | | CCR5-TS6, |
| | hCCR5- | 21 | gcgtcatcccaagagtctct | CCR5-TS7, |
| | 2-1R | | | CCR5-TS8 |
| | hCCR5- | 22 | ACACTCTTTCCCTACACGACGCTCTT | |
| | 2-deepF | | CCGATCTtttgcgtctctcccaggaat | |
| | hCCR5- | 23 | GTGACTGGAGTTCAGACGTGTGCTC | |
| | 2-deepR | | TTCCGATCTgatggtgaagataagcctcaca | |
| DNMT1 | hDNMT | 24 | ttgggaatgtgggtactgct | DNMT1-TS1, |
| | 1-1-1F | | | DNMT1-TS2, |
| | hDNMT | 25 | cagaggttacagtgagccga | DNMT1-TS3, |
| | 1-1-1R | | | DNMT1-TS4 |
| | hDNMT | 26 | ACACTCTTTCCCTACACGACGCTCTT | |
| | 1-1- | | CCGATCTTCTtgtgthccttgttctctgacac | |
| | deepF | | | |
| | hDNMT | 27 | GTGACTGGAGTTCAGACGTGTGCTC | |
| | 1-1- | | TTCCGATCTgtttggaaaggggtttgggg | |
| | deepR | | | |
| | hDNMT | 28 | taggcatgtaccaccacacc | DNMT1-TS5, |
| | 1-2-1F | | | DNMT1-TS6, |
| | hDNMT | 29 | tcagtgcccaccgataatca | DNMT1-TS7, |
| | 1-2-1R | | | DNMT1-TS8 |
| | hDNMT | 30 | ACACTCTTTCCCTACACGACGCTCTT | |
| | 1-2- | | CCGATCTtgtcaagtgggtgatctct | |
| | deepF | | | |
| | hDNMT | 31 | GTGACTGGAGTTCAGACGTGTGCTC | |
| | 1-2- | | TTCCGATCTtgtagaacttgttgacccgga | |
| | deepR | | | |

### Example 4: 6TG Selection

After transfection of the AacC2c1 vector and the sgRNA vector thereinto in 24-well-plate scales, the cells were passaged to secure a sufficient number. One week after transfection, 1.5X₁06 cells were seeded in a 100 pi dish. From the next day, selection was started in a 6 μM 6TG (6-thioguanine) condition. Two weeks later, colonies of the cells in which the HPRT1 gene had been knocked out (KO) by mutation were identified by crystal violet staining.

### Example 5: Confirmation of AacC2c1 Expression

Prior to genome editing, examination was made to see whether the AacC2c1 protein was expressed in human cells. In this regard, the pcDNA3.1-AacC2c1-NLS-HA plasmid was transfected into HEK293T cells with the aid of lipofectamine2000 (see Example 4). Twenty four hours after transfection, a whole cell lysate (WCL) was taken and assayed for AacC2c1 expression by a Western blotting method using a 1^{st} antibody specifically recognizing an HA tag present at the C-terminus of the AacC2c1 protein so as to specifically observe only the AacC2c1 protein.

The results are depicted in FIG. 1b (left panel: 1^{st} antibody, HA tag; right panel: 1^{st} antibody, GAPDH). As shown in FIG. 1b, when the 1^{st} antibody specifically recognizing an HA tag was used, a protein was detected at 133 kDa in the p3-Aac-C2c1-CN plasmid-transfected experiment group, but not in the mock control.

### Example 6: Genome Editing Using AacC2c1

Because the AacC2c1 protein was identified to be expressed in human cells (HEK293T cells) in Example 5, an examination was made to test whether human genome editing could be conducted with AacC2c1. A human HPRT1 gene was selected as a target gene. Experimental preference was given to HPRT1 gene because when complete KO was introduced by gene editing, a KO clone could be identified on a phenotype basis through 6-thioguanine (6-TG) selection and crystal violet staining.

AacC2c1 target sequences were selected for human HPRT1 gene and exon sequences of CCR5 and DNMT1 genes (Table 2), and respective Aac-sgRNA vectors corresponding to the target sequences were constructed (see Example 1).

TABLE 2

| | | SEQ |
|---|---|---|
| | TARGET SITE | ID |
| | (5′-nTTN(nPAM)-X₂0) | NO |
| AacC2c1-HPRT1-TS1 | tTTTGCATACCTAATCATTATGCT | 32 |
| AacC2c1-HPRT1-TS2 | aTTATGCTGAGGATTTGGAAAGGG | 33 |
| AacC2c1-HPRT1-TS3 | aTTAGGTATGCAAAATAAATCAAG | 34 |
| AacC2c1-HPRT1-TS4 | tTTCCAAATCCTCAGCATAATGAT | 35 |
| AacC2c1-HPRT1-TS5 | aTTGTAGCCCTCTGTGTGCTCAAG | 36 |
| AacC2c1-HPRT1-TS6 | tTTGCTGACCTGCTGGATTACATC | 37 |
| AacC2c1-HPRT1-TS7 | cTTGAGCACACAGAGGGCTACAAT | 38 |
| AacC2c1-HPRT1-TS8 | tTTGATGTAATCCAGCAGGTCAGC | 39 |
| AacC2c1-CCR5-TS1 | tTTCCTTCTTACTGTCCCCTTCTG | 40 |
| AacC2c1-CCR5-TS2 | cTTACTGTCCCCTTCTGGGCTCAC | 41 |
| AacC2c1-CCR5-TS3 | aTTTCCAAAGTCCCACTGGGCGGC | 42 |
| AacC2c1-CCR5-TS4 | gTTGACACATTGTATTTCCAAAGT | 43 |
| AacC2c1-CCR5-TS5 | tTTCCAGACATTAAAGATAGTCAT | 44 |
| AacC2c1-CCR5-TS6 | aTTAAAGATAGTCATCTTGGGGCT | 45 |
| AacC2c1-CCR5-TS7 | cTTGTCATGGTCATCTGCTACTCG | 46 |
| AacC2c1-CCR5-TS8 | gTTTTTAGGATTCCCGAGTAGCAG | 47 |
| AacC2c1-DNMT1-TS1 | tTTGTCCTTGGAGAACGGTGCTCA | 48 |
| AacC2c1-DNMT1-TS2 | gTTCTCCAAGGACAAATCTTTATT | 49 |
| AacC2c1-DNMT1-TS3 | cTTACAACCGGGAAGTGAATGGAC | 50 |
| AacC2c1-DNMT1-TS4 | aTTCACTTCCCGGTTGTAAGCATG | 51 |
| AacC2c1-DNMT1-TS5 | tTTCCGGTAGTGCTCTGGGTACAG | 52 |
| AacC2c1-DNMT1-TS6 | gTTGCTGCCTTTGATGTAGTCGGA | 53 |
| AacC2c1-DNMT1-TS7 | aTTGGCCGGATCAAAGAGATCTTC | 54 |
| AacC2c1-DNMT1-TS8 | cTTCTGTCCCAAGAAGAGCAACGG | 55 |

(X₂0 regions corresponding to the sequences except n(PAM) account for target sequences)

AacC2c1 and Aac-sgRNA expression vectors were delivered into Hela cells and HEK293T cells with the aid of lipofectamine2000. 72 Hours after vector delivery, genomic DNA was isolated, and analyzed by targeted deep sequencing for gene editing efficiency on on-target sequences (introduced indel frequency (%)). For assessing the statistical significance of the introduced indels, targeted deep sequencing was performed on mock controls, which had not been treated with the AacC2c1 and Aac-sgRNA expression vectors. The experiment procedures were repeated three times in total.

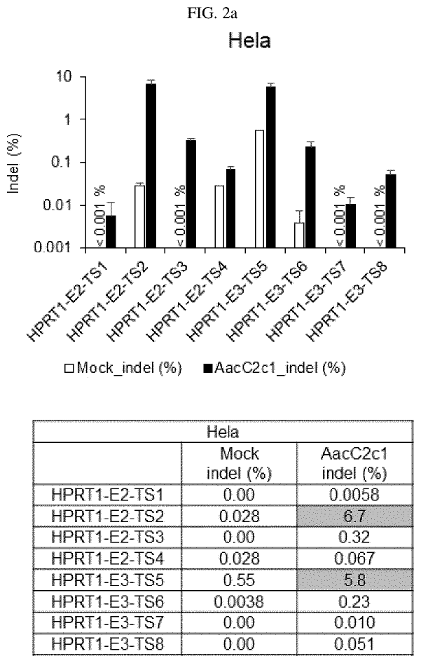

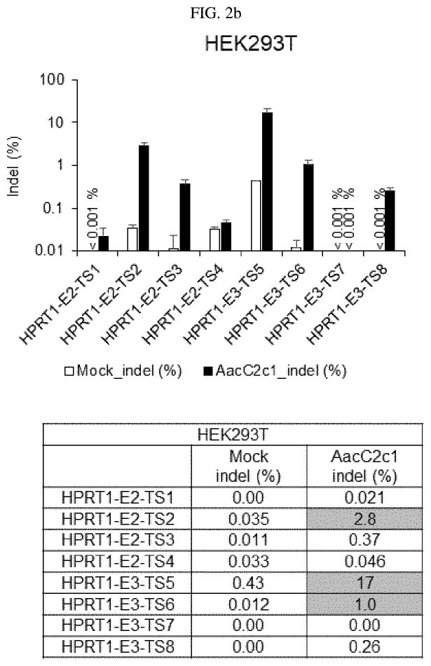

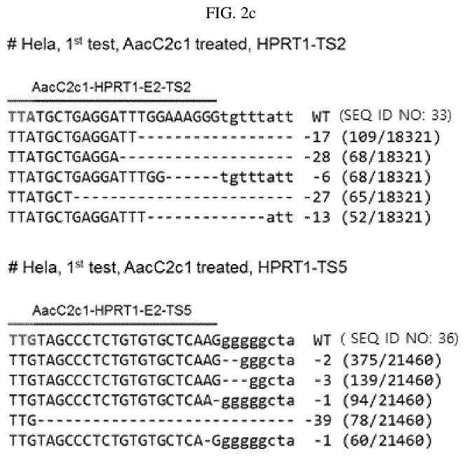

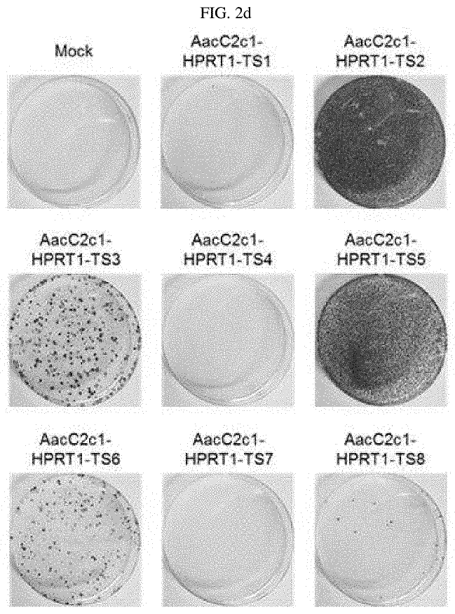

Analysis results are depicted in FIGS. 2a (Hela cell) and 2b (HEK293T cell). As shown in FIGS. 2a and 2b, HPRT1-TS2 and HPRT1-TS5 were respectively observed to introduce indel at frequencies of as high as 6.7% and 5.8% on average into the on-target sequences of Hela cells and at frequencies of as high as 2.8% and 17% on average into the target-sequences of HEK293T cells.

In addition, targeted deep sequencing raw data were analyzed to examine how indels were mainly introduced into on-target sequences. The results are depicted in FIG. 2c. As can be seen in FIG. 2c, the mutation pattern is deletion that was made mainly at 3′ terminal regions of the on-target sequences (FIG. 2c).

In order to more clearly prove the results obtained by the genetic analysis, the Hela cells to which the AacC2c1 and Aac-sgRNA expression vectors were delivered were subjected to 6-TG selection and crystal violet staining to investigate HPRT1 gene-knockout cells on a phenotype basis. In greater detail, the AacC2c1 and sgRNA expression vectors were transfected into Hela cells which were then seeded and subjected to 6-thioguanine (6-TG) selection. Two weeks later, colonies of the cells in which HPRT1 gene was knocked out by mutation were detected by crystal violet staining. The results are depicted in FIG. 2d. As shown in FIG. 2d, the cells to which the Aac-sgRNA and AacC2c1 plasmids corresponding respectively to HPRT1-TS2 and HPRT1-TS5 were delivered had the HPRT1 gene completely knocked out therein, and thus formed a lot of colonies as detected by 6-TG selection and appeared intensively stained upon crystal violet staining

Examination was made to investigate whether the AacC2c1-CRISPR-Cas endonucleases could make gene editing on target sequences of genes other than HPRT1 gene. To this end, CCR5 and DNMT1 genes were additionally selected. For each of the target genes, 8 AacC2c1 target sequences were selected (Table 2). Aac-sgRNA vectors corresponding respectively to the target sequence were delivered, together with the AacC2c1 expression plasmid, into Hela cells and HEK293T cells. 72 hours after delivery, genomic DNA was extracted and analyzed for the introduction of indels into on-target sequences to measure indel frequencies.

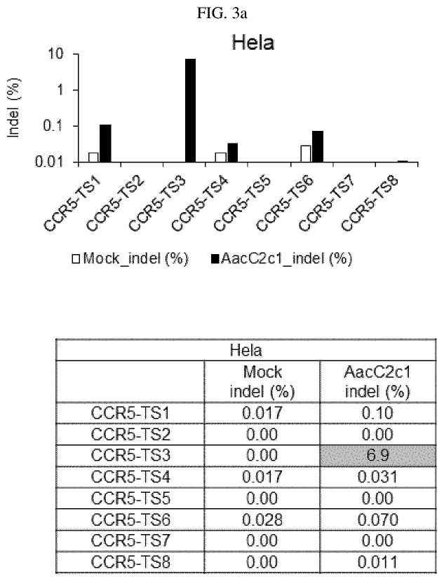

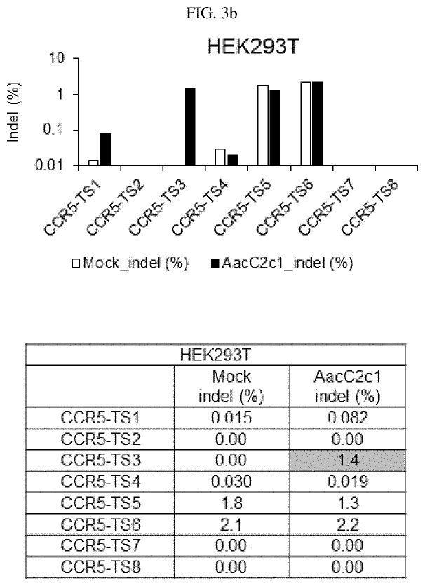

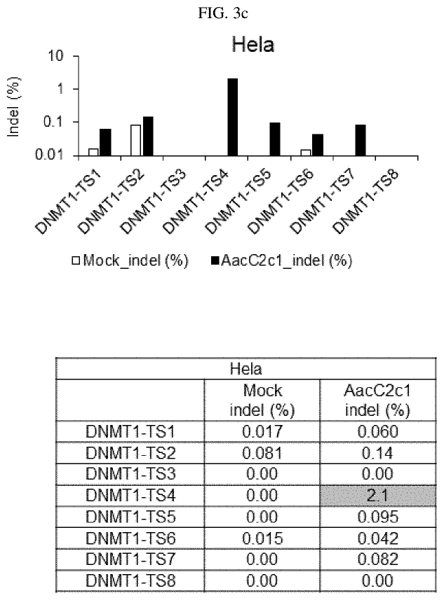

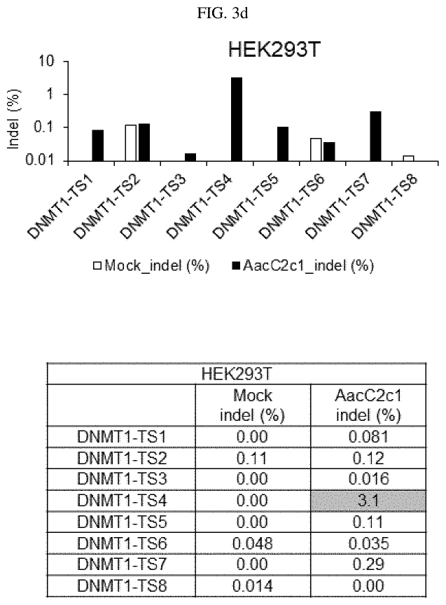

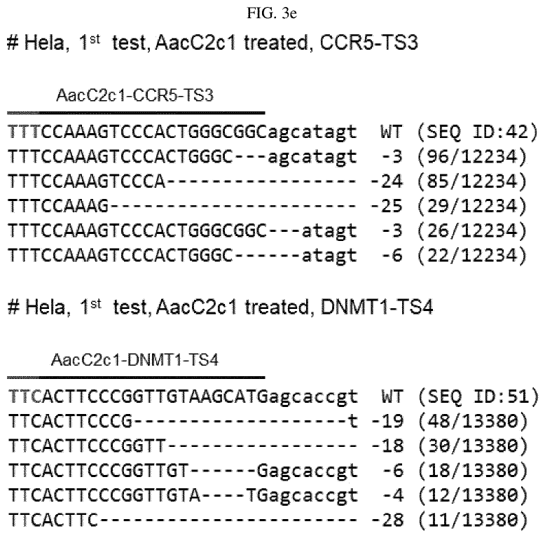

The indel frequencies thus obtained are depicted in FIGS. 3a (indel frequencies on CCR5 target regions in Hela cells), 3b (indel frequencies on CCR5 target regions in HEK293T cells), 3c (indel frequencies on DNMT1 target regions in Hela cells), and 3d (indel frequencies on DNMT1 target regions in HEK293T cells). As can be seen in FIGS. 3a to 3d, indels were introduced into the CCR5-TS3 target region at frequencies of 6.9% for Hela and 1.4% for HEK293T cells and into the DNMT1-TS4 region at frequencies of 2.1% for Hela cells and 3.1% for HEK293T cells.

In addition, 1^{st} NGS (targeted deep sequencing) was performed for CCR5-TS3 and DNMT1-TS4 and the result is depicted in FIG. 3e. As shown in FIG. 3e, both CCR5-TS3 and DNMT1-TS4 were observed to have indels introduced into the target sequences therein.

Based on phenotype analysis results, genetic analysis confirmed that the wild-type AacC2c1-CRISPR system can be used for human genome editing and operates for specific target sequence locations at high yield.

1. A genome editing composition, comprising:
a C2c1 endonuclease, a gene coding therefor, or an expression vector carrying the gene; and
a guide RNA, a DNA coding therefor, or an expression vector carrying the DNA.

2. The genome editing composition of claim 1, wherein the C2c1 endonuclease is derived from Alicyclobacillus acidoterrestris.

3. The genome editing composition of claim 2, wherein the guide RNA is represented by the following General Sequence Formula 1 or is a mutant resulting from the deletion of 1-15 nucleotides from the sequence of General Sequence Formula 1:
5′-GUCUAGAGGACAGAAUUUUUCAACGGGUGUGCCAAUGGCCAC
UUUCCAGGUGGCAAAGCCCGUUGAGCUUCUCAAAUCUGAGAAGUG
GX₁X₂X₃[(N)n]-3′
(General Sequence Formula 1)
wherein,
X₁, X₂, and X₃ are each a nucleotide independently selected from A, U, G, and C, and
(N)n stands for n nucleotides that may be the same or different and are each independently selected from A, U, G, and C, wherein n is an integer of 17-23.

4. The genome editing composition of claim 3, wherein the deletion is performed on 1-15 nucleotides selected from the sequence AGCUUCUCAAA in General Sequence Formula 1.

5. The genome editing composition of claim 3, wherein the guide RNA is represented by the following General Sequence Formula 2:
5′-GUCUAGAGGACAGAAUUUUUCAACGGGUGUGCCAAUGGCCAC
UUUCCAGGUGGCAAAGCCCGUUGAGCUUCUCAAAUCUGAGAAGUG
GCAC[(N)n]-3′
(General Sequence Formula 2)
wherein,
(N)n represents n nucleotides which may be the same or different and are selected from A, U, G, and C wherein n is an integer of 17-23.

6. The genome editing composition of claim 5, wherein n is 20.

7. The genome editing composition of claim 1, wherein the C2c1 endonuclease further comprises a nuclear localization signal.

8. The genome editing composition of claim 1, wherein the genome editing composition is applied to an eukaryotic cell or an eukaryotic organism.

9. A genome editing method, comprising a step of introducing the genome editing composition of claim 1 into an isolated cell or organisms exclusive of humans.

10. The genome editing method of claim 9, wherein the cell is an eukaryotic cell and the organism is an eukaryotic organism exclusive of humans.

11. An isolated genetically modified cell, having the genome editing composition of claim 1 introduced thereinto.

12. A genetically modified organism, exclusive of a human, having the genome editing composition of claim 1 introduced thereinto.
